(19) United States
(12) Patent Application Publication
(10) Pub. No.: US 2013/0144145 A1
(43) Pub. Date: Jun. 6, 2013

(54) IMPLANTABLE NEURAL TISSUE REPORTING PROBE AND METHODS OF MANUFACTURING AND IMPLANTING SAME

(71) Applicant: UNIVERSITY OF SOUTHERN CALIFORNIA, Los Angeles, CA (US)

(72) Inventor: Ellis Meng, Alhambra, CA (US)

(73) Assignee: UNIVERSITY OF SOUTHERN CALIFORNIA, Los Angeles, CA (US)

(21) Appl. No.: 13/693,838

(22) Filed: Dec. 4, 2012

Related U.S. Application Data
(60) Provisional application No. 61/566,906, filed on Dec. 5, 2011.

(51) Int. Cl. A61B 5/04

(52) U.S. Cl. CPC A61B 5/04001
USPC 600/377; 264/272.14

(57) ABSTRACT

A method of manufacturing an implantable neural tissue reporting probe may include affixing multiple electrodes to polymeric material; heating the polymeric material to a temperature that is above its glass transition temperature, but below its melting temperature; applying force to the polymeric material while heated so as to cause the polymeric material to change into a shape that is suitable for implanting in neural tissue, the shape including a compartment having at least one opening therein sized to permit dendritic growth to occur through the opening from outside of the compartment to within the compartment after the probe is implanted; and allowing the polymeric material to cool down below its glass transition temperature while maintaining the shape of the compartment, including while maintaining the shape of the opening therein. Related probes and methods of implanting them into neural tissue are also disclosed.

### CROSS-REFERENCE TO RELATED APPLICATION

This application is based upon and claims priority to U.S. provisional patent application 61/566,906, entitled “THREE-DIMENSIONAL HOLLOW ELECTRODES AND METHOD TO MANUFACTURE THREE-DIMENSIONAL STRUCTURES,” filed Dec. 5, 2011, attorney docket number 028080-0699. The entire content of this application is incorporated herein by reference.

### STATEMENT REGARDING FEDERALLY SPONSORED RESEARCH

This invention was made with government support under Grant No. N66001-11-1-4207, awarded by the Defense Advanced Research Projects Agency (DARPA). The Government has certain rights in the invention.

### BACKGROUND

1. Technical Field

This disclosure relates to implantable neural tissue reporting probes and to methods of manufacturing and implanting the same.

2. Description of Related Art

Today's implant technologies may be limited in their ability to treat multiple neurological disorders and injuries in wounded war fighters, as well as in others. In order to use action potentials of cortical neurons as control signals for a brain-machine interface, implanted microelectrodes may need to be both reliable and have a stable interface with the neural tissue. However, the ability of chronic microelectrodes to record resolvable neuronal activities may be reduced or completely lost over time. Gradual retraction of the dendritic tree may degrade the recording quality of intracortical microelectrodes.

Such dendritic neurodegeneration may be caused by neurotoxic factors released by microglia due to chronic ongoing inflammatory response close to the microelectrodes aggravated by a mechanical mismatch between the rigid probe and the cortical tissue. See McConnell, G. C., H. D. Rees, A. I. Levey, C. A. Gutekunst, R. E. Gross, and R. V. Bellamkonda, Implanted neural electrodes cause chronic, local inflammation that is correlated with local neurodegeneration. J Neural Eng, 2009. 6(5): p. 056003; Winslow, B. D., M. B. Christensen, W.-K. Yang, F. Solzbacher, and P. A. Tresco, A comparison of the tissue response to chronically implanted Parylene-C-coated and uncoated planar silicon microelectrode arrays in rat cortex. Biomaterials, 2010. 31(35): p. 9163-9172; and Winslow, B. D. and P. A. Tresco, Quantitative analysis of the tissue response to chronically implanted microwire electrodes in rat cortex. Biomaterials, 2010. 31(7): p. 1558-1567.

One approach to improving the long-term reliability of the cortical interface in the recording of well-resolved neuronal action potentials has been to design the electrode to attract the dendritic processes into the electrode vicinity and to apply several coatings to the electrode. Three dimensional (3D) hollow shafts have been decorated with multiple microelectrodes and have provided a high density recording interface with neural tissue. The shaft interior and/or exterior has been coated with neurotrophic factors, neuronal-survival promoting factors, anti-inflammatory compounds, and/or other agents to enhance the connection and promote long-term reporting reliability. The neurotrophic factors may provide encouragement to the ingrowth of dendritic processes towards this end. However, it may be difficult to manufacture such devices.

Implantable cortical electrodes have enjoyed decades of development, but few have been successfully implemented in a human and, even so, with only a short device lifetime (e.g., <5 years). See Bartels, J., D. Andreasen, P. Ehirim, H. Mao, S. Seibert, E. J. Wright, and P. Kennedy, Neurotrophic electrode: method of assembly and implantation into human motor speech cortex. J Neurosci Methods, 2008. 174(2): p. 168-76; Guenther, F. H., J. S. Brumberg, E. J. Wright, A. Nieto-Castanon, J. A. Tourville, M. Panko, R. Law, S. A. Siebert, J. L. Bartels, D. S. Andreasen, P. Ehirim, H. Mao, and P. R. Kennedy, A wireless brain-machine interface for real-time speech synthesis. PLoS One, 2009. 4(12): p. e8218; Kennedy, P., Comparing Electrodes for use as Cortical Control Signals: Tiny Tines, Tiny Wires or Tiny Cones on Wires: Which is best?, in The Biomedical Engineering Handbook, J. Brazino, Editor. 2006. p. 32-1 to 32.14; Kennedy, P., D. Andreasen, P. Ehirim, B. King, T. Kirby, H. Mao, and M. Moore, Using human extra-cortical local field potentials to control a switch. Journal of Neural Engineering, 2004. 1(2): p. 72; Kennedy, P. R. and R. A. Bakay, Restoration of neural output from a paralyzed patient by a direct brain connection. Neuroreport, 1998. 9(8): p. 1707-11; Suner, S., M. R. Fellows, C. Vargas-Irwin, G. K. Nakata, and J. P. Donoghue, Reliability of signals from a chronically implanted, silicon-based electrode array in non-human primate primary motor cortex. IEEE Trans Neural Syst Rehabil Eng, 2005. 13(4): p. 524-41; Polikov, V. S., P. A. Tresco, and W. M. Reichert, Response of brain tissue to chronically implanted neural electrodes. Journal of Neuroscience Methods, 2005. 148: p. 1-18; and Ryu, S. I. and K. V. Shenoy, Human cortical prostheses: lost in translation? Neurosurg Focus, 2009. 27(1): p. E5.

Two very different approaches to establishing an electrical interface have been demonstrated in humans to have long recording lifetimes:

(1) Donoghue group used an array of tapered-tip silicon pins each with an individual electrode at the tip, see Suner, S., M. R. Fellows, C. Vargas-Irwin, G. K. Nakata, and J. P. Donoghue, Reliability of signals from a chronically implanted, silicon-based electrode array in non-human primate primary motor cortex. IEEE Trans Neural Syst Rehabil Eng, 2005. 13(4): p. 524-41; Maynard, E. M., C. T. Nordhausen, and R. A. Normann, The Utah intracortical Electrode Array: a recording structure for potential brain-computer interfaces. Electroencephalogr Clin Neurophysiol, 1997. 102(3): p. 228-39; Campbell, P. K., K. E. Jones, R. J. Huber, K. W. Horch, and R. A. Normann, A silicon-based, three-dimensional neural interface: manufacturing processes for an intracortical electrode array. IEEE Trans Biomed Eng, 1991. 38(8): p. 758-68; Hochberg, L. R., M. D. Serruya, G. M. Friehs, J. A. Mukand, M. Saleh, A. H. Caplan, A. Branner, D. Chen, R. D. Penn, and J. P. Donoghue, Neuronal ensemble control of prosthetic devices by a human with tetraplegia. Nature, 2006. 442(7099): p. 164-71; and

(2) Kennedy group used individual hollow glass cones with 2-4 wires with de-insulated tips on the interior, see Bartels, J., D. Andreasen, P. Ehirim, H. Mao, S. Seibert, E. J. Wright, and P. Kennedy, Neurotrophic electrode: method of assembly and implantation into human motor speech cortex. J Neurosci Methods, 2008. 174(2): p. 168-76; Guenther, F. H., J. S. Brumberg, E. J. Wright, A. Nieto-Castanon, J. A. Tourville, M. Panko, R. Law, S. A. Siebert, J. L. Bartels, D. S. Andreasen, P. Ehirim, H. Mao, and P. R. Kennedy, A wireless brain-machine interface for real-time speech synthesis. PLoS One, 2009. 4(12): p. e8218; Kennedy, P., Comparing Electrodes for use as Cortical Control Signals: Tiny Tines, Tiny Wires or Tiny Cones on Wires: Which is best?, in The Biomedical Engineering Handbook, J. Brazino, Editor. 2006. p. 32-1 to 32.14; Kennedy, P., D. Andreasen, P. Ehirim, B. King, T. Kirby, H. Mao, and M. Moore, Using human extra-cortical local field potentials to control a switch. Journal of Neural Engineering, 2004. 1(2): p. 72; Kennedy, P. R. and R. A. Bakay, Restoration of neural output from a paralyzed patient by a direct brain connection. Neuroreport, 1998. 9(8): p. 1707-11; Kennedy, P. R., The cone electrode: a long-term electrode that records from neurites grown onto its recording surface. J Neurosci Methods, 1989. 29(3): p. 181-93; Kennedy, P. R., R. A. Bakay, and S. M. Sharpe, Behavioral correlates of action potentials recorded chronically inside the Cone Electrode. Neuroreport, 1992. 3(7): p. 605-8; and Kennedy, P., Implantable Neural Electrode. 1989: United States.

In the latter, neurotrophic factors encouraged ingrowth of dendritic processes into the cone (˜3 months).

Overall, degradation of recording quality in implanted neural electrodes is due to many factors several of which have been addressed by rational design: biocompatibility, mechanical stiffness mismatch, geometry, size, texture, and bioactive coatings. See Suner, S., M. R. Fellows, C. Vargas-Irwin, G. K. Nakata, and J. P. Donoghue, Reliability of signals from a chronically implanted, silicon-based electrode array in non-human primate primary motor cortex. IEEE Trans Neural Syst Rehabil Eng, 2005. 13(4): p. 524-41; Polikov, V. S., P. A. Tresco, and W. M. Reichert, Response of brain tissue to chronically implanted neural electrodes. Journal of Neuroscience Methods, 2005. 148: p. 1-18; and Ward, M. P., P. Rajdev, C. Ellison, and P. P. Irazoqui, Toward a comparison of microelectrodes for acute and chronic recordings. Brain Res, 2009. 1282: p. 183-200.

### SUMMARY

A method of manufacturing an implantable neural tissue reporting probe may include affixing multiple electrodes to polymeric material; heating the polymeric material to a temperature that is above its glass transition temperature, but below its melting temperature; applying force to the polymeric material while heated so as to cause the polymeric material to change into a shape that is suitable for implanting in neural tissue, the shape including a compartment having at least one opening therein sized to permit dendritic growth to occur through the opening from outside of the compartment to within the compartment after the probe is implanted; and allowing the polymeric material to cool down below its glass transition temperature while maintaining the shape of the compartment, including while maintaining the shape of the opening therein.

The polymeric material may be deposited onto a substrate before the heating. The polymeric material may be released from the substrate before the heating or after the cooling.

Multiple layers of the polymeric material may be deposited onto the substrate before the heating.

Two or more of the deposited layers of polymeric material may form a pocket. The applying force may include inserting a tool into the pocket, thereby forming the compartment and the opening.

The substrate may be substantially flat.

The affixing multiple electrodes to the piece of polymeric material may include depositing the electrodes on the polymeric material after the polymeric material has been deposited on the substrate and before the polymeric material has been released from the substrate.

The electrodes may be affixed to the polymeric material before the polymeric material is heated.

The polymeric material may be Parylene C. The polymeric material may be another thermoplastic polymer that will soften but not burn during the heating.

The compartment may have multiple openings, each sized to permit dendritic growth to occur through the opening from outside of the compartment to within the compartment after the probe is implanted.

The compartment may have a conical or cylindrical shape.

At least one of the electrodes may be within the compartment.

At least one of the electrodes may be outside of the compartment.

A method of implanting the implantable neural tissue reporting probe into neural tissue may include inserting a tool into a compartment through an opening; applying longitudinal force to the tool in the direction of the neural tissue so as to cause the implantable neural tissue reporting probe to be implanted into the neural tissue; and removing the tool from the compartment. The method of implanting may include mounting the probe adjacent to a tool using a biodegradable adhesive; applying a longitudinal force to the tool in the direction of the neural tissue so as to cause the implantable neural tissue reporting probe to be implanted into the neural tissue; and removing the tool from the compartment after the adhesive has dissolved.

These, as well as other components, steps, features, objects, benefits, and advantages, will now become clear from a review of the following detailed description of illustrative embodiments, the accompanying drawings, and the claims.

### BRIEF DESCRIPTION OF DRAWINGS

The drawings are of illustrative embodiments. They do not illustrate all embodiments. Other embodiments may be used in addition or instead. Details that may be apparent or unnecessary may be omitted to save space or for more effective illustration. Some embodiments may be practiced with additional components or steps and/or without all of the components or steps that are illustrated. When the same numeral appears in different drawings, it refers to the same or like components or steps.

FIG. 1 illustrates an example of a system that uses neural signals detected by an implantable neural tissue reporting probe to drive a prosthetic limb.

FIG. 2A illustrates an example of an implantable neural tissue reporting probe. FIG. 2B illustrates multiple implantable neural tissue reporting probes, each slidably mounted on a prong of an introducer tool.

FIG. 3A illustrates an example of an implantable neural tissue reporting probe in which exterior electrodes are on top of a surface of a compartment in an implantable neural tissue reporting probe. FIG. 3B illustrates an embodiment of an implantable neural tissue reporting probe in which exterior electrodes are to the side of a compartment in the implantable neural tissue reporting probe.

FIGS. 4A-4G illustrate an example of a process for manufacturing an implantable neural tissue reporting probe.

FIGS. 5A-5G illustrate an example of a process for manufacturing a different type of implantable neural tissue reporting probe.

FIGS. 6A1-6C2 illustrate an example of a thermoforming process that may be used to create a 3D compartment.

FIG. 7 illustrates an example of a thermoforming fixture that may include of a microwire inserted into a Parylene channel to form a cone shaped compartment in an implantable neural tissue reporting probe.

FIG. 8A illustrates an example of a three dimensional neural probe formed by sequential thermoforming processes that form the cone tip first, followed by a strain-relief coil. FIG. 8B illustrates an SEM image of an example of the thermoformed cone illustrated in FIG. 8A.

FIG. 9A illustrates an example of a three dimensional neural probe that has a conical shape with perforations through the sheath, shown with a thermoforming microwire positioned in the lumen of the cone. FIG. 9B illustrates an example of a cylindrical shaped neural probe, also with perforations through the sheath.

FIG. 10 illustrates an example of a compartment in an implantable neural tissue reporting probe that is filled with polyethylene glycol (PEG, clear) and that has a blunt microwire attached using PEG.

### DETAILED DESCRIPTION OF ILLUSTRATIVE EMBODIMENTS

Illustrative embodiments are now described. Other embodiments may be used in addition or instead. Details that may be apparent or unnecessary may be omitted to save space or for a more effective presentation. Some embodiments may be practiced with additional components or steps and/or without all of the components or steps that are described.

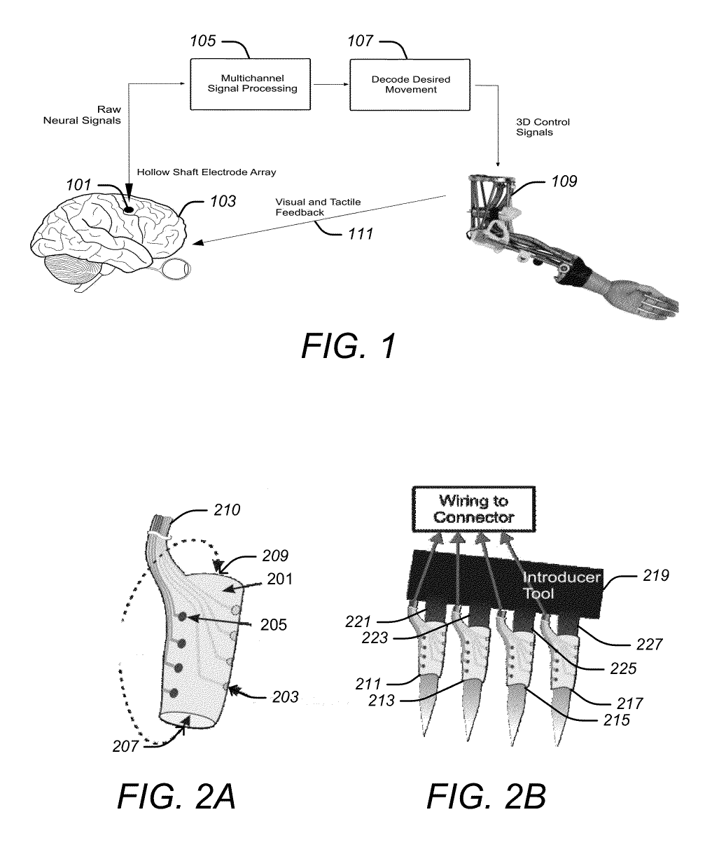

FIG. 1 illustrates an example of a system that uses neural signals detected by an implantable neural tissue reporting probe to drive a prosthetic limb. As illustrated in FIG. 1, an implantable neural tissue reporting probe 101 may be implanted in neural tissue, such as in a brain 103. The signals from the neural tissue reporting probe 101 may be processed by a multi-channel signal processing system 105, decoded into desired movements by a decode desired movement decoder 107, and used to drive a robotically-controlled prosthetic device, such as a robotically-controlled arm 109. Visual feedback 111 may be used by the brain 103 to generate feedback signals to the probe that are processed and decoded to make needed adjustments.

FIG. 2A illustrates an example of an implantable neural tissue reporting probe. As illustrated in FIG. 2, the probe may include a compartment in the form of a hollow compartment 201 that includes one or more internal electrodes, such an internal electrode 203; one or more external electrodes, such as an external electrode 205; one or more openings, such as openings 207 and 209; and a flexible ribbon cable 210 containing conducting leads to the electrodes, which may be embedded in a polymeric material, such as Parylene C. The compartment 201 may be in the shape of sheath.

The electrodes, such as the electrodes 203 and 205, may be patterned on the compartment 201 using microfabrication techniques both on the interior and exterior surface of the shaft 201. Some of these electrodes may be reserved for self-testing in order to monitor the reliability of the tissue-electrode interface over time. The interior and/or exterior of the compartment 203 may be coated with one or more neurotrophic factors, neuronal-survival promoting factors, anti-inflammatory compounds, and/or other agents to enhance the connection and/or promote long-term reliability. The neurotrophic factors may provide encouragement to the ingrowth of dendritic processes.

FIG. 2B illustrates multiple implantable neural tissue reporting probes 211, 213, 215, and 217, each slidably mounted, respectively, on a prong 221, 223, 225, and 227, of an introducer tool 219. The tool may be moved longitudinally in the direction of neural tissue, thereby causing each of the implantable neural tissue reporting probes to be simultaneously implanted into the neural tissue. Thereafter, the introducer tool may be removed, thereby causing each of the implantable neural tissue reporting probes to slide off of the prong to which they are slidably mounted, thereby leaving the implantable neural tissue reporting probes implanted in the neural tissue. A different number and/or configuration of the probes and their associated prongs may be used instead.

Thermal modification of polymer structures, referred to herein as thermoforming, may be achieved by heating a thermoplastic polymer above its glass transition temperature, but below its melting point. While heated to this temperature, its shape may be adjusted. The heat may be removed and the adjusted shape may be retained. See Truckenmuller, R., S. Giselbrecht, N. Rivron, E. Gottwald, V. Saile, A. van der Berg, M. Wessling, and C. van Blitterswijk, Thermoforming of Film-Based Biomedical Microdevices. Advanced Materials, 2011. 23: p. 1311-1329.

Within the thermoforming temperature range, polymeric chains may be free to move and undergo thermally induced reorganization. See Davis, E. M., N. M. Benetatos, W. F. Regnault, K. I. Winey, and Y. A. Elabd, The influence of thermal history of structure and water transport in Parylene C coatings. Polymer, 2011. 52: p. 5378-5386. This process may use a mechanical mold and /or pressure to facilitate the shaping.

It can be extremely difficult to achieve three dimensional shapes using common microfabrication processes due to the planar, layer-by-layer nature in which structural materials are processed. As a result, microfabrication usually produces largely flat, planar structures.

By thermoforming the flat structures produced by microfabrication, three dimensional structures with far greater utility can be achieved. This process can have great utility in the creation of compartments that can be part of implantable neural tissue reporting probes.

To achieve a stable long-term interface and sufficient recording sites, for example, to improve an achievable number of degrees of freedom (to drive motor prostheses), and to perform self-testing, an implantable neural tissue reporting probe may combine the advantages of neurotrophic cone electrodes with multisite silicon shanks. Hollow polymeric compartments can be formed into 3D shapes (e.g. cylindrical or conical) and may contain a high density of planar electrodes decorating both the interior and exterior of the compartment, as illustrated in FIGS. 2A and 2B. This electrode arrangement may maximize accessible recording units and provide extra channels for self-testing of probe performance. The hollow interface structure may allow ingrowth of dendritic processes for stable, long-term recordings and may secure electrodes in the tissue. This strategy may take advantage of microfabrication processes for batch fabrication of complex 3D structures and may offer a manufacturable pathway for human use.

The individual implantable neural tissue reporting probes may not be rigidly bound together, such as with a superstructure, and may be implanted with the aid of an introducer tool, as illustrated in FIG. 2B. As such, they may not have to be as long as cortical depth like traditional silicon probes. To maximize reliability, the implantable neural tissue reporting probe may contain external and internal cavity coatings (e.g. neurotrophic, neuronal survival-promoting, anti-inflammatory). The coatings may either be applied directly to the compartment 201 (with appropriate formulations to adjust the duration and speed of release) or be released slowly over time through integrated microfluidic channels connected to a reservoir that may be refillable. The microfluidic channel system may include integrated pumps, valves, and sensors to regulate and monitor the speed and duration of delivery. The compartment 201 may be appropriately perforated with outlets to spread the agents to the tissue.

Microfabrication is a process in which microstructures may be created using planar processes that are either additive or subtractive. Technological limitations may result in the creation of structures that are largely planar.

Batch fabrication of complex 3D hollow shaft cortical interfaces with electrode sites on both sides may be enabled by biocompatible Parylene micromachining.

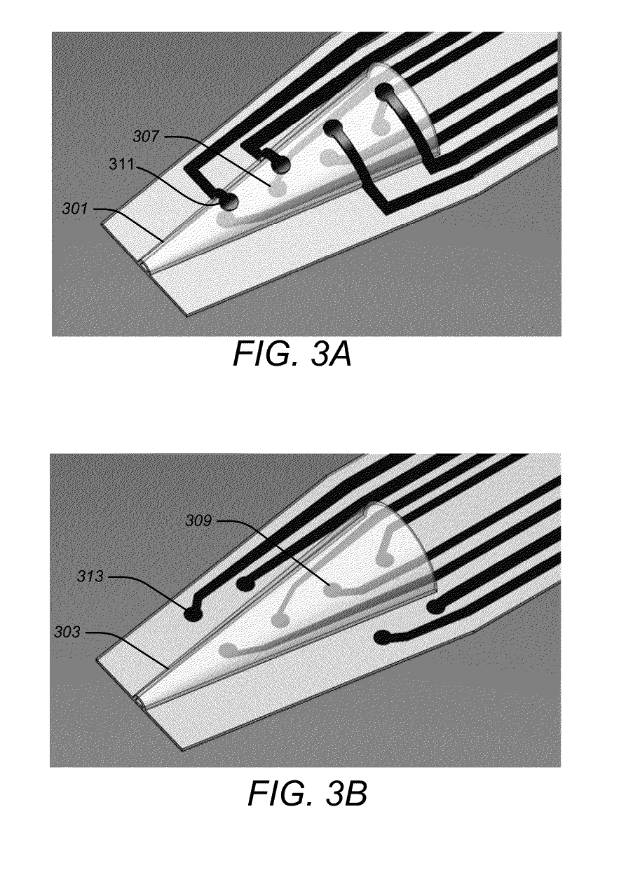

FIGS. 3A and 3B illustrate an example of two Parylene sheath probes that may facilitate long-term intracortical recordings. Each probe may include a 3D sheath compartment 301 or 303 that allows for ingrowth of neural processes toward the recording electrodes. Each probe may include internal electrodes within the sheath compartment 301 or 303, such as internal electrodes 307 and 309. FIG. 3A illustrates an embodiment in which exterior electrodes are on top of the outer sheath compartment surface, such as an external electrode 311. FIG. 3B illustrates an embodiment in which exterior electrodes are to the side of the sheath compartment, such as an external electrode 313.

Parylene surface micromachining processes may be used to fabricate planar structures initially supported by rigid substrates, such as those illustrated in FIGS. 3A and 3B. See Rodger, D. C., A. J. Fong, L. Wen, H. Ameri, A. K. Ahuja, C. Gutierrez, I. Lavrov, Z. Hui, P. R. Menon, E. Meng, J. W. Burdick, R. R. Roy, V. R. Edgerton, J. D. Weiland, M. S. Humayun, and Y. C. Tai, Flexible parylene-based multielectrode array technology for high-density neural stimulation and recording. Sensors and Actuators B-Chemical, 2008. 132(2): p. 449-460; Li, W., D. C. Rodger, E. Meng, J. D. Weiland, M. S. Humayun, and Y. C. Tai, Wafer-level Parylene Packaging with Integrated RF Electronics for Wireless Retinal Prosthesis. IEEE/ASME Journal of Microelectromechanical Systems, 2010. 19(4): p. 735-742; Gutierrez, C. A., C. Lee, B. Kim, and E. Meng. Epoxy-less Packaging Methods for Electrical Contact to Parylene-based Flat Flexible Cables. in The 16th International Conference on Solid-State Sensors, Actuators and Microsystems, IEEE Transducers. 2011, (accepted). Beijing, China; Meng, E., P. Y. Li, and Y. C. Tai, Plasma removal of parylene c. Journal of Micromechanics and Microengineering, 2008. 18(4); Gutierrez, C. A., C. McCarty, B. Kim, M. Pahwa, and E. Meng. An Implantable All-Parylene Liquid-Impedance based MEMS Force Sensor. in IEEE MEMS. 2010. Hong Kong, China p. 600-603; Gutierrez, C. A. and E. Meng. A Dual Function Parylene-based Biomimetic Tactile Sensor and Actuator for Next Generation Mechanically Responsive Microelectrode Arrays. in The 15th International Conference on Solid-State Sensors, Actuators and Microsystems, IEEE Transducers. 2009. Denver, Colo., USA p. 2194-2197; Gutierrez, C. A. and E. Meng, Parylene-based Electrochemical-MEMS Transducers. J. Microelectromech. Sys., 2010. 19(6): p. 1352-1361; Gutierrez, C. A. and E. Meng. Fabrication of a Parylene-based Microforce Sensor Array for an Epiretinal Prosthesis. in 39th Neural Interfaces Conference. 2010. Long Beach, Calif., USA p. 142; Gutierrez, C. A. and E. Meng. A Subnanowatt Microbubble Pressure Transducer. in Hilton Head Workshop: A Solid-State Sensors, Actuators and Microsystems Workshop. 2010. Hilton Head Island, S.C., USA p. 57-60; and Gutierrez, C. A. and E. Meng. A Subnanowatt Microbubble Pressure Sensor based on Electrochemical Impedance Transduction in a Flexible All-Parylene Package. in IEEE MEMS. 2011. Cancun, Mexico p. 549-552.

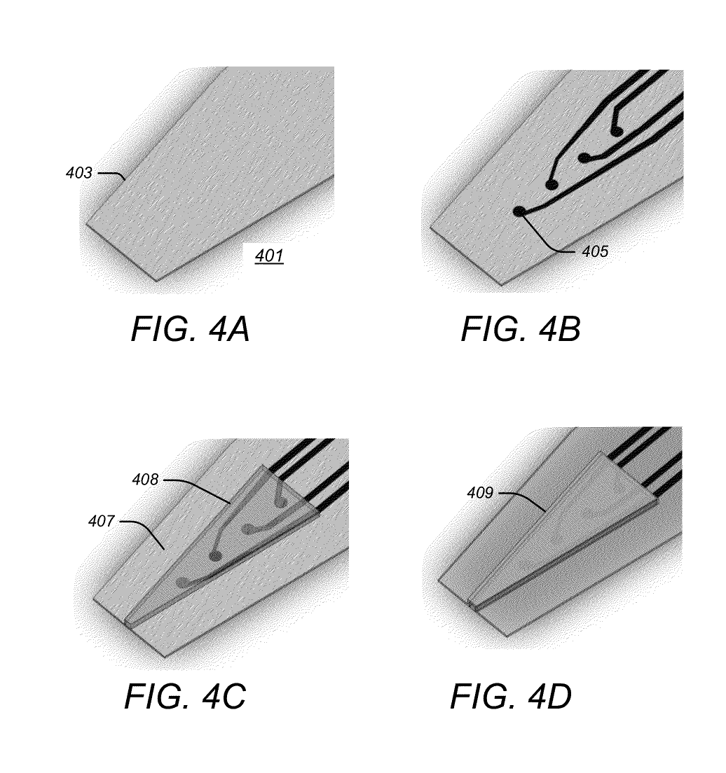

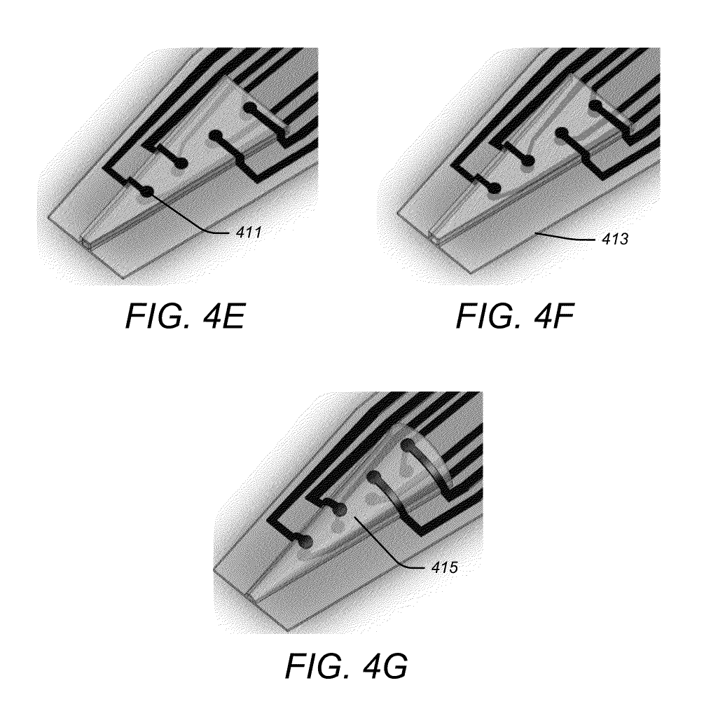

FIGS. 4A-4G illustrate an example of a fabrication process for sheath probes having electrodes on the top of the outer sheath compartment surface. (The image sequence is simplified and the drawings only capture the final outline of the device.) As illustrated in these figures and as discussed in more detail below, hollow cylindrical or cone structures may be formed by thermoforming following release and sacrificial material removal, with high reproducibility and precision.

The thermoforming process may include holding a polymer structure in a fixture that maintains the final desired shape while subjecting the whole assembly to elevated temperatures. The process may be carried out under vacuum to eliminate oxidative processes that may damage the polymer. Following the thermal process (performed above but near the glass transition temperature), the polymer may retain the new shape and the guide fixture may be removed.

Reliability may be enhanced by facilitating ingrowth of dendritic processes by using hollow shaft electrodes and neurotrophic factor coatings. The electrode count may be increased; a batch fabrication process may be used for the shaft electrodes; and the coating types applied to such a structure can be increased. Dedicated electrode sites on the interior and exterior surfaces may be included for self-testing of overall electrode reliability over time.

The shaft electrodes may be arrayable for maximizing recording inputs and accessible brain volume. Implantation of arrays with a custom introducer tool may allow reliable placement into the cortex. Shaft electrodes may be easily scaled up and may be fabricated using processes that avoid manufacturing inconsistencies in hand-made glass cone or wire electrodes. Other approaches that enjoyed success with neurotrophic factor-mediated ingrowth of dendritic processes may also be used. See Bartels, J., D. Andreasen, P. Ehirim, H. Mao, S. Seibert, E. J. Wright, and P. Kennedy, Neurotrophic electrode: method of assembly and implantation into human motor speech cortex. J Neurosci Methods, 2008. 174(2): p. 168-76; Guenther, F. H., J. S. Brumberg, E. J. Wright, A. Nieto-Castanon, J. A. Tourville, M. Panko, R. Law, S. A. Siebert, J. L. Bartels, D. S. Andreasen, P. Ehirim, H. Mao, and P. R. Kennedy, A wireless brain-machine interface for real-time speech synthesis. PLoS One, 2009. 4(12): p. e8218; Kennedy, P., Comparing Electrodes for use as Cortical Control Signals: Tiny Tines, Tiny Wires or Tiny Cones on Wires: Which is best?, in The Biomedical Engineering Handbook, J. Brazino, Editor. 2006. p. 32-1 to 32.14; Kennedy, P., D. Andreasen, P. Ehirim, B. King, T. Kirby, H. Mao, and M. Moore, Using human extra-cortical local field potentials to control a switch. Journal of Neural Engineering, 2004. 1(2): p. 72; Kennedy, P. R. and R. A. Bakay, Restoration of neural output from a paralyzed patient by a direct brain connection. Neuroreport, 1998. 9(8): p. 1707-11; Kennedy, P. R., The cone electrode: a long-term electrode that records from neurites grown onto its recording surface. J Neurosci Methods, 1989. 29(3): p. 181-93; Kennedy, P. R., R. A. Bakay, and S. M. Sharpe, Behavioral correlates of action potentials recorded chronically inside the Cone Electrode. Neuroreport, 1992. 3(7): p. 605-8; Benfey, M. and A. J. Aguayo, Extensive elongation of axons from rat brain into peripheral nerve grafts. Nature, 1982. 296(5853): p. 150-152; David, S. and A. J. Aguayo, Axonal elongation into peripheral nervous system “bridges” after central nervous system injury in adult rats. Science, 1981. 214(4523): p. 931-933; David, S. and A. J. Aguayo, Axonal regeneration after crush injury of rat central nervous system fibres innervating peripheral nerve grafts. 1985(1): p. 1-12; and Sugar, O. and R. W. Gerard, Spinal Cord Regeneration in the Rat. J. Neurophysiol., 1940. 3: p. 1-19.

The hollow structure may allow coating of interior and exterior surfaces. The interior coatings may encourage ingrowth, while exterior coatings may promote neuronal survival and suppress inflammatory response, thereby improving long term recording reliability. The coatings may include nerve growth factor (NGF), brain-derived neurotrophic factor (BDNF), neurotrophin-3 (NT-3), neurotrophin-4 (NT-4), ciliary neurotrophic factor (CNTF), glial cell line-derived neurotrophic factor (GDNF), and/or dexamethasone.

In one embodiment, Parylene C may be used for the structure and platinum may be used for the electrodes. Parylene C may be biocompatible and may serve as an insulation layer for the neural electrodes. See Rodger, D. C., A. J. Fong, L. Wen, H. Ameri, A. K. Ahuja, C. Gutierrez, I. Lavrov, Z. Hui, P. R. Menon, E. Meng, J. W. Burdick, R. R. Roy, V. R. Edgerton, J. D. Weiland, M. S. Humayun, and Y. C. Tai, Flexible parylene-based multielectrode array technology for high-density neural stimulation and recording. Sensors and Actuators B-Chemical, 2008. 132(2): p. 449-460; Li, W., D. C. Rodger, E. Meng, J. D. Weiland, M. S. Humayun, and Y. C. Tai, Wafer-level Parylene Packaging with Integrated RF Electronics for Wireless Retinal Prosthesis. IEEE/ASME Journal of Microelectromechanical Systems, 2010. 19(4): p. 735-742; and Gutierrez, C. A., C. Lee, B. Kim, and E. Meng. Epoxy-less Packaging Methods for Electrical Contact to Parylene-based Flat Flexible Cables. in The 16th International Conference on Solid-State Sensors, Actuators and Microsystems, IEEE Transducers. 2011, Beijing, China p. 2299-2302. Other polymers, such as polydimethylsiloxane, polyimide, other Parylenes, and/or polymethylmethacrylate may instead be used as the structural material. Instead of platinum, any other conductive material may be used, such as a metal, metal oxide, conductive polymer, silicon derivative, or combinations thereof. If a metal is used, it may include one or more of the following platinum derivatives or alloys (such as platinum grey or black or Ptlr), gold, iridium, titanium, chromium, copper, aluminum, tungsten, silver, silver chloride, indium tin oxide, iridium oxide, or any combinations thereof.

The three dimensional structures illustrated in FIGS. 3A and 3B may be used in recording neural activity within the brain.

FIGS. 4A-4G illustrate an example of a process for manufacturing the implantable neural tissue reporting probe illustrated in FIG. 3A. The overall structure may be fabricated using Parylene C, a thin film thermoplastic polymer, and the electrodes from a suitable biocompatible metal, such as Pt. Other polymers and conductive materials may be used in addition or instead.

A bare Si wafer 401 with native oxide may be used as a carrier substrate during the microfabrication process and aided in the subsequent release of Parylene probes from the wafer by lifting off or peeling.

Sheath probes having sheath-top electrodes may be fabricated by first depositing a pattern 403 of μm Parylene on the substrate, as illustrated in FIG. 4A. A liftoff process using negative photoresist (AZ 5214 E-IR) may be utilized to pattern inner sheath electrodes, such as inner sheath electrode 405, created with e-beam deposited Pt (2000 Å), as illustrated in FIG. 4B. A 1 μm Parylene insulation layer 407 may then be deposited and selectively plasma etched to expose the inner electrodes and contact pads, as illustrated in FIG. 4C. Sheath outlines may be constructed by patterning sacrificial photoresist (AZ 4620, 9.6 μm) 408, as illustrated in FIG. 4C, and overcoating with a pattern 409 of 5 μm Parylene, as illustrated in FIG. 4D. A dual layer liftoff scheme (AZ 1518/AZ 4620) with negative sidewall profile may be utilized to pattern outer electrodes on top of the sheath structure. This may help ensure that resulting wire traces are continuous from the top of the microchannel structure to the base. See Gutierrez, C. A. and E. Meng, Parylene-based Electrochemical-MEMS Transducers. J. Microelectromech. Sys., 2010. 19(6): p. 1352-1361; and Gutierrez, C. A. and E. Meng, A dual function Parylene-based biomimetic tactile sensor and actuator for next generation mechanically responsive microelectrode arrays, in The 15th International Conference on Solid-State Sensors, Actuators and Microsystems (TRANSDUCERS). 2009, IEEE: Denver, Colo., USA, p. 2194-2197. Pt may then be e-beam deposited (2000 Å) to form the outer electrodes, such as outer electrode 411 as illustrated in FIG. 4E. A final 1 μm Parylene insulation layer 413 may be deposited and plasma etched to create openings for outer electrodes and contact pads, as illustrated in FIG. 4F. A final plasma etch may be performed to create sheath openings and cut out the individual probes.

Probes may be released from the substrate by lifting off or gentle peeling, and the sacrificial photoresist may be removed with an acetone soak. A micromould may then be inserted into the pocket formed by the multiple overlapping layers of Parylene and thermoformed to obtain the desired 3D structure 415, as illustrated in FIG. 4G.

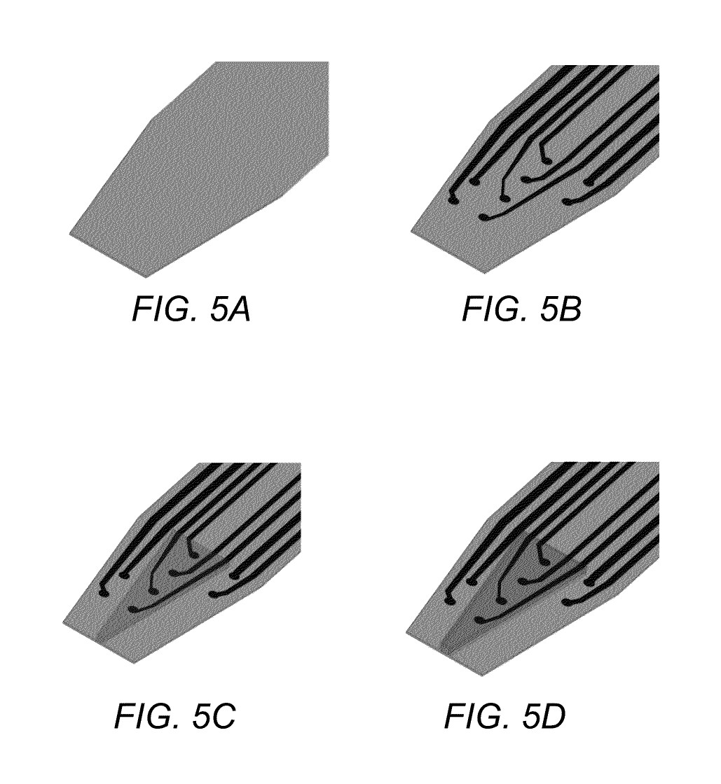

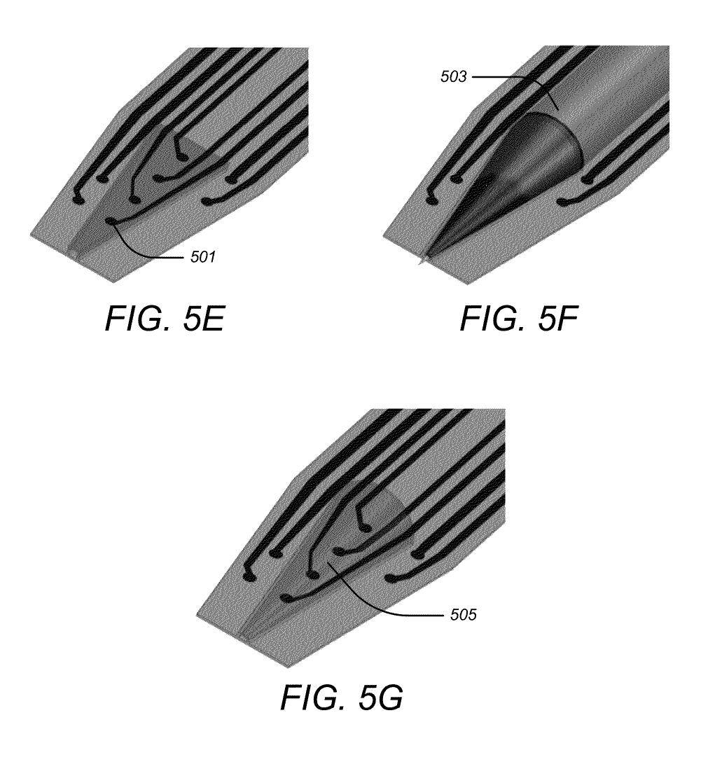

FIGS. 5A-5G illustrate an example of a process for manufacturing a different type of implantable neural tissue reporting probe. The steps may be the same as are illustrated in FIGS. 4A-4G and as described above, except that the outer electrodes, such as an outer electrode 501 in FIG. 4E, may be moved to the periphery. This may reduce the number of steps required and may also prevent occasional cracking of the top electrodes that might otherwise be encountered during the sheath forming process. FIG. 5F also illustrates insertion of a micromould 503 inserted into the pocket formed by the multiple overlapping layers of Parylene and thermoformed to obtain the desired 3D compartment structure 505, as illustrated in FIG. 5G.

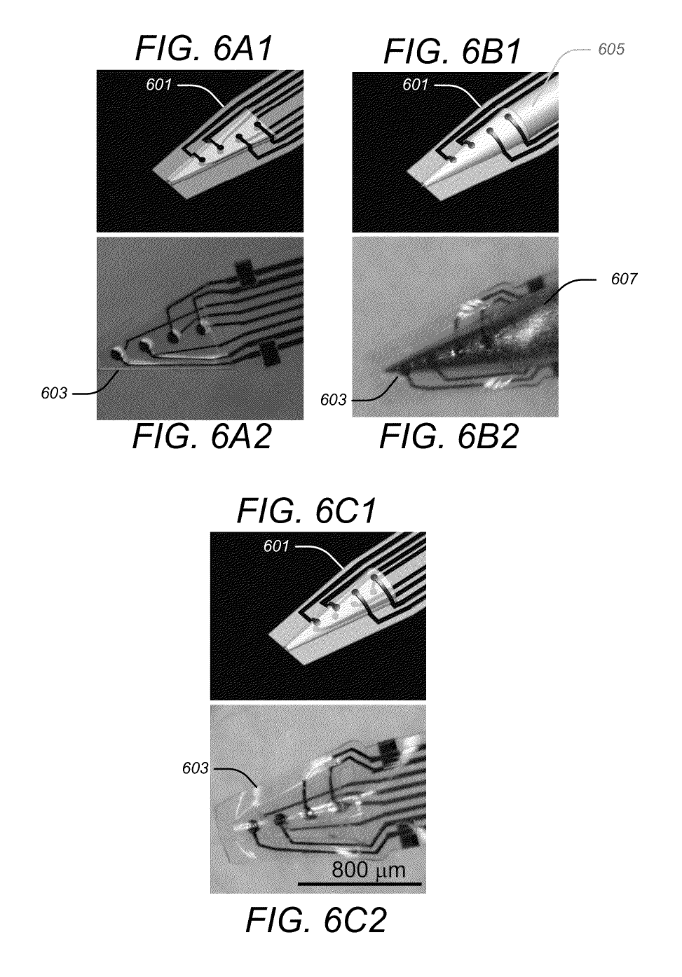

FIGS. 6A1-6C2 illustrate an example of a thermoforming process that may be used to create a 3D compartment structure. The figures with a “1” suffix are theoretical drawings, while those with a “2” suffix are photographs of a corresponding probe that was actually fabricated. The process may be used for the steps illustrated in FIGS. 4G, 5F, and 5G.

FIGS. 6A1 and 6A2 illustrate a released probe 601 and 603, respectively, containing a microchannel compartment structure. This may be shaped using a microwire mold 605 and 607, respectively, to prop open the channel and subsequently thermoformed to lock in the structure, as illustrated in FIGS. 6B1 and 6B2. Subsequently, the wire mold 605 and 607 may be removed to reveal the final structure, as illustrated in FIGS. 6C1 and 6C2.

Conical or cylindrical 3D sheath structures may be created by thermoforming Parylene around a custom tapered stainless steel or tungsten microwire mold. Etched microwires with tapers to match the desired probe shape and to facilitate may be inserted into the microchannels. A microwire tip may be aligned and inserted into the sheath underneath a microscope to open the structure. The assembly may be held in an aluminum fixture and placed into a vacuum oven. Thermoforming may be performed with a controlled temperature ramp to 200° C. and held for 48 hours, followed by a controlled cool down. Nitrogen purging may prevent Parylene oxidative degradation by minimizing oven oxygen content. After cooling, the microwire may then be removed and the sheath may retain its 3D structure.

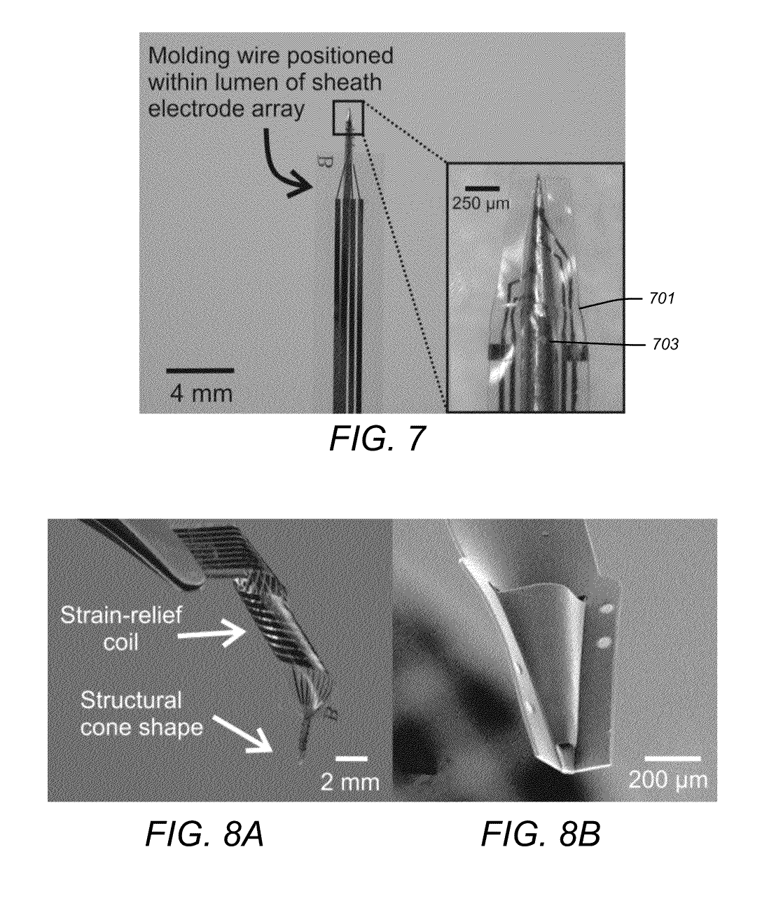

FIG. 7 illustrates an example of a thermoforming fixture 701. A microwire 703 may be inserted into a Parylene channel to form cone shaped sheath electrodes for neural recordings. The microwire may be positioned in the lumen of the cone to maintain the final desired shape during the thermal treatment.

FIG. 8A illustrates an example of a three dimensional neural probe formed by sequential thermoforming processes that formed the cone tip first, followed by a strain-relief coil. FIG. 8B illustrates an SEM image of an example of the thermoformed cone illustrated in FIG. 8A. These shaping and thermoforming steps can be repeated sequentially to achieve complex three dimensional shapes, as illustrated in FIG. 8A.

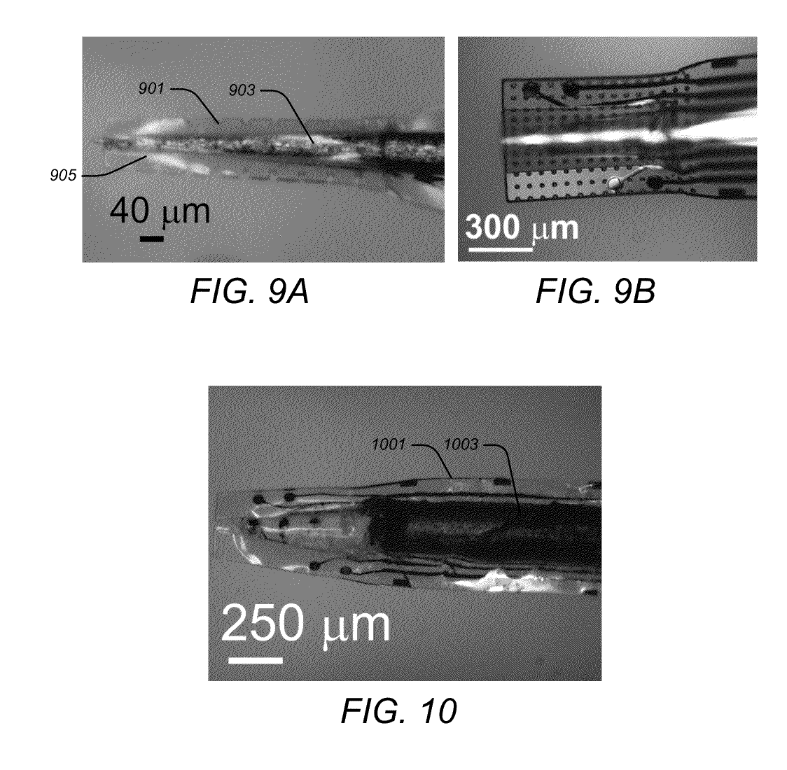

FIG. 9A illustrates an example of a three dimensional neural probe that has a conical shape with perforations through the sheath, such as a perforation 901, shown with a thermoforming microwire 903 positioned in the lumen of the cone 905. FIG. 9B illustrates an example of a cylindrical shaped neural probe, also with perforations through the sheath. As illustrated in FIGS. 9A and 9B, the three dimensional structures may include additional perforations to further attract ingrowth of dendritic processes or promote tissue integration.

Direct incorporation of integrated circuits, discrete electronic components, and even RF coils with the shaft electrodes is possible with the Parylene technology that has been described. See Li, W., D. C. Rodger, E. Meng, J. D. Weiland, M. S. Humayun, and Y. C. Tai, Wafer-level Parylene Packaging with Integrated RF Electronics for Wireless Retinal Prosthesis. IEEE/ASME Journal of Microelectromechanical Systems, 2010. 19(4): p. 735-742; Gutierrez, C. A., C. Lee, B. Kim, and E. Meng. Epoxy-less Packaging Methods for Electrical Contact to Parylene-based Flat Flexible Cables. in The 16th International Conference on Solid-State Sensors, Actuators and Microsystems, IEEE Transducers. 2011, Beijing, China p. 2299-2302; and Li, W., D. C. Rodger, E. Meng, J. D. Weiland, M. S. Humayun, and Y. C. Tai, Flexible Parylene Packaged Intraocular Coil for Retinal Prosthesis, in International Conference on Microtechnologies in Medicine and Biology. 2006: Okinawa, Japan. p. 105-108 and related polymer technologies.

FIG. 10 illustrates an example of a sheath compartment 1001 filled with polyethylene glycol (PEG, clear) and with a blunt microwire 1003 also attached using PEG. The wire may be used to push the assembly into neural tissue, such as a brain. PEG is a water soluble wax that may dissolve after the probe is implanted, allowing the wire to be retracted.

Individual or arrays of hollow electrode shafts can be implanted through the use of a custom introducer tool, such as the one illustrated in FIG. 2B. Each shaft may be matched to a rigid probe that provides structural support during inserting of the shaft into neural tissue. After insertion, the tool may be removed, leaving the shaft electrode in the tissue.

Two embodiments of the sheath probes were designed, fabricated, and demonstrated in neural recordings from rat brains. These probes were constructed using thermoforming to create the sheath compartment portion of the probe. Sequential thermoforming was performed to create strain relief structures in the cable attached to the probes, such as is illustrated in FIG. 8A. The probes were implanted using a variety of temporary stiffeners, such as the one illustrated in FIGS. 9A and 9B.

In summary, a method has been described for fabricating three-dimensional compartment structures using a combination of microfabrication processes, post-fabrication assembly, and thermoforming. The three dimensional compartment structure may be formed from a thermoplastic material amenable to thermoforming. The three dimensional compartment structure may contain other materials that cannot be thermoformed, but serve other purposes on the final structure. The post-fabrication assembly process may be assisted by the use of an intermediate shaping mold to hold the part in its final intended shape during the thermoforming process. The method may be repeated such that a part may be shaped sequentially to achieve a final three dimensional desired structure.

A method for fabricating three dimensional structures may have electrode sites decorating the interior and exterior surfaces. The three dimensional structure may be formed from a thermoplastic material amenable to thermoforming. The three dimensional structure may contain other materials that cannot be thermoformed but serve other purposes on the final structure. The hollow three dimensional structures may be constructed of biocompatible materials suited for long term implantation in the body; may serve as an interface to tissue; may contain electrodes used for recording and/or stimulation of neural tissues or muscle; may contain sensory elements for interacting with tissue; may contain biomolecule and drug-eluting coatings on its surfaces; may be hollow to attract ingrowth of dendritic processes or otherwise integrate with tissue; may include perforations along the hollow structure to promote ingrowth of dendritic processes and/or integration with tissue; may contain additional electrodes or sensory elements for self-testing and diagnostic purposes; may be arranged in an array; and/or may be implanted using a tool or coating to provide temporary stiffness during penetration of tissues.

A method for implanting the hollow shaft electrodes may use an introducer tool. The tool may be inserted into the compartment through the opening in the compartment. Longitudinal force may be applied to the tool in the direction of the neural tissue so as to cause the implantable neural tissue reporting probe to be implanted into the neural tissue. The tool may then be removed from the compartment and the tissue.

The components, steps, features, objects, benefits, and advantages that have been discussed are merely illustrative. None of them, nor the discussions relating to them, are intended to limit the scope of protection in any way. Numerous other embodiments are also contemplated. These include embodiments that have fewer, additional, and/or different components, steps, features, objects, benefits, and advantages. These also include embodiments in which the components and/or steps are arranged and/or ordered differently.

For example, the method of implanting may include mounting the probe adjacent to a tool using a biodegradable adhesive; applying a longitudinal force to the tool in the direction of the neural tissue so as to cause the implantable neural tissue reporting probe to be implanted into the neural tissue; and removing the tool from the compartment after the adhesive has dissolved. The electrodes affixed to the polymer may be replaced with or accompanied by sensor elements that gather additional physiological information from the biological surroundings adjacent to the implanted probe. Some or all of the electrodes affixed to the polymer may alternatively be used for stimulation of tissue. The sizing of the electrodes, whether or stimulation or reporting, should be sized and placed appropriately according to the target tissue anatomy.

Unless otherwise stated, all measurements, values, ratings, positions, magnitudes, sizes, and other specifications that are set forth in this specification, including in the claims that follow, are approximate, not exact. They are intended to have a reasonable range that is consistent with the functions to which they relate and with what is customary in the art to which they pertain.

All articles, patents, patent applications, and other publications that have been cited in this disclosure are incorporated herein by reference.

The phrase “means for” when used in a claim is intended to and should be interpreted to embrace the corresponding structures and materials that have been described and their equivalents. Similarly, the phrase “step for” when used in a claim is intended to and should be interpreted to embrace the corresponding acts that have been described and their equivalents. The absence of these phrases from a claim means that the claim is not intended to and should not be interpreted to be limited to these corresponding structures, materials, or acts, or to their equivalents.

The scope of protection is limited solely by the claims that now follow. That scope is intended and should be interpreted to be as broad as is consistent with the ordinary meaning of the language that is used in the claims when interpreted in light of this specification and the prosecution history that follows, except where specific meanings have been set forth, and to encompass all structural and functional equivalents.

Relational terms such as “first” and “second” and the like may be used solely to distinguish one entity or action from another, without necessarily requiring or implying any actual relationship or order between them. The terms “comprises,” “comprising,” and any other variation thereof when used in connection with a list of elements in the specification or claims are intended to indicate that the list is not exclusive and that other elements may be included. Similarly, an element preceded by an “a” or an “an” does not, without further constraints, preclude the existence of additional elements of the identical type.

None of the claims are intended to embrace subject matter that fails to satisfy the requirement of Sections 101, 102, or 103 of the Patent Act, nor should they be interpreted in such a way. Any unintended coverage of such subject matter is hereby disclaimed. Except as just stated in this paragraph, nothing that has been stated or illustrated is intended or should be interpreted to cause a dedication of any component, step, feature, object, benefit, advantage, or equivalent to the public, regardless of whether it is or is not recited in the claims.

The abstract is provided to help the reader quickly ascertain the nature of the technical disclosure. It is submitted with the understanding that it will not be used to interpret or limit the scope or meaning of the claims. In addition, various features in the foregoing detailed description are grouped together in various embodiments to streamline the disclosure. This method of disclosure should not be interpreted as requiring claimed embodiments to require more features than are expressly recited in each claim. Rather, as the following claims reflect, inventive subject matter lies in less than all features of a single disclosed embodiment. Thus, the following claims are hereby incorporated into the detailed description, with each claim standing on its own as separately claimed subject matter.

The invention claimed is:

1. A method of manufacturing an implantable neural tissue reporting probe comprising:
affixing multiple electrodes to polymeric material;
heating the polymeric material to a temperature that is above its glass transition temperature, but below its melting temperature;
applying force to the polymeric material while heated so as to cause the polymeric material to change into a shape that is suitable for implanting in neural tissue, the shape including a compartment having at least one opening therein sized to permit dendritic growth to occur through the opening from outside of the compartment to within the compartment after the probe is implanted; and
allowing the polymeric material to cool down below its glass transition temperature while maintaining the shape of the compartment, including while maintaining the shape of the opening therein.

2. The method of manufacturing an implantable neural tissue reporting probe of claim 1 further comprising:
depositing the polymeric material onto a substrate before the heating; and
releasing the polymeric material from the substrate before the heating or after the cooling.

3. The method of manufacturing an implantable neural tissue reporting probe of claim 2 wherein the depositing the polymeric material onto the substrate before the heating includes depositing multiple layers of the polymeric material onto the substrate before the heating.

4. The method of manufacturing an implantable neural tissue reporting probe of claim 3 wherein:
two or more of the deposited layers of polymeric material form a pocket; and
the applying force includes inserting a tool into the pocket, thereby forming the compartment and the opening.

5. The method of manufacturing an implantable neural tissue reporting probe of claim 2 wherein the substrate is substantially flat.

6. The method of manufacturing an implantable neural tissue reporting probe of claim 2 wherein the affixing multiple electrodes to the piece of polymeric material includes depositing the electrodes on the polymeric material after the polymeric material has been deposited on the substrate and before the polymeric material has been released from the substrate.

7. The method of manufacturing an implantable neural tissue reporting probe of claim 1 wherein the electrodes are affixed to the polymeric material before the polymeric material is heated.

8. The method of manufacturing an implantable neural tissue reporting probe of claim 1 wherein the polymeric material is Parylene C.

9. The method of manufacturing an implantable neural tissue reporting probe of claim 1 wherein the compartment has multiple openings, each sized to permit dendritic growth to occur through the opening from outside of the compartment to within the compartment after the probe is implanted.

10. The method of manufacturing an implantable neural tissue reporting probe of claim 1 wherein the compartment has a conical or cylindrical shape.

11. An implantable neural tissue reporting probe comprising:
polymeric material that has a shape that is suitable for implanting in neural tissue, the shape including a compartment having at least one opening therein sized to permit dendritic growth to occur through the opening from outside of the compartment to within the compartment after the probe is implanted; and
multiple electrodes attached to the polymeric material.

12. The implantable neural tissue reporting probe of claim 11 wherein the compartment includes multiple openings, each sized to permit dendritic growth to occur through the opening from outside of the compartment to within the compartment after the probe is implanted.

13. The implantable neural tissue reporting probe of claim 11 wherein the polymeric material is Parylene C.

14. The implantable neural tissue reporting probe of claim 11 wherein at least one of the electrodes is within the compartment.

15. The implantable neural tissue reporting probe of claim 14 wherein at least one of the electrodes is outside of the compartment.

16. The implantable neural tissue reporting probe of claim 11 further comprising one or more sensors configured to gather physiological information from biological surroundings adjacent the probe after it is implanted in neural tissue, in addition to neural signals.

17. The implantable neural tissue reporting probe of claim 11 further comprising a coating on the polymeric material that is configured to slowly release into neural tissue and to reduce inflammatory response, enhance the tissue-electrode connection, and/or promote long-term reporting reliability.

18. The implantable neural tissue reporting probe of claim 11 further comprising a fluidic conduit that elutes or pumps one or more liquid agents into neural tissue that reduce inflammatory response, enhance the tissue-electrode connection, and/or promote long-term reporting reliability.

19. An implantable neural tissue reporting probe comprising:
material that has a shape that is suitable for implanting in neural tissue, the shape including a compartment having at least one opening therein sized to permit dendritic growth to occur through the opening from outside of the compartment to within the compartment after the probe is implanted; and
multiple electrodes attached to the material, at least one of which is within the compartment and at least one of which is outside of the compartment.

20. The implantable neural tissue reporting probe of claim 19 wherein the at least one electrode that is attached outside of the compartment is attached to the exterior of the compartment.

21. The implantable neural tissue reporting probe of claim 19 wherein the at least one electrode that is attached outside of the compartment is attached to the material at a location that is not the exterior of the compartment.

22. A method of implanting an implantable neural tissue reporting probe into neural tissue, the reporting probe including material that has a shape that is suitable for implanting in neural tissue, the shape including a compartment having at least one opening therein sized to permit dendritic growth to occur through the opening from outside of the compartment to within the compartment after the probe is implanted, the reporting probe further including multiple electrodes attached to the material, the method comprising in the order recited:
inserting a tool into the compartment through the opening;
applying longitudinal force to the tool in the direction of the neural tissue so as to cause the implantable neural tissue reporting probe to be implanted into the neural tissue; and
removing the tool from the compartment.

23. The method of claim 22 wherein the compartment has a conical or cylindrical shape.
